(19) United States
(12) Patent Application Publication
(10) Pub. No.: US 2023/0183161 A1
(43) Pub. Date: Jun. 15, 2023

(54) CONTINUOUS PROCESS TO MAKE TRIFLUOROACETYL IODIDE FROM TRIFLUOROACETYL CHLORIDE AND HYDROGEN IODIDE BY REACTIVE DISTILLATION

(71) Applicant: Honeywell International Inc., Charlotte, NC (US)

(72) Inventors: Terris Yang, East Amherst, NY (US); Haiyou Wang, Amherst, NY (US); Christian Jungong, Depew, NY (US)

(21) Appl. No.: 18/075,936

(22) Filed: Dec. 6, 2022

Related U.S. Application Data
(60) Provisional application No. 63/289,461, filed on Dec. 14, 2021.

(51) Int. Cl. C07C 51/363

(52) U.S. Cl. CPC C07C 51/363

(57) ABSTRACT

The present disclosure provides a process for producing trifluoroacetyl iodide (TFAI) from trifluoroacetyl chloride (TFAC) and hydrogen iodide (HI) via reactive distillation. The process may be conducted in the presence or absence of a catalyst. The process may be conducted in the presence or absence of a solvent.

### CROSS-REFERENCE TO RELATED APPLICATION

This application claims priority to U.S. Provisional Application No. 63/289,461, filed Dec. 14, 2021, which is herein incorporated by reference in its entirety.

### FIELD

The present disclosure provides continuous processes for producing trifluoroacetyl iodide (TFAI) from trifluoroacetyl chloride (TFAC) and hydrogen iodide (HI) via a continuous reactive distillation which combines the reactor and HCl removal column into one apparatus. The present disclosure further provides an integrated TFAI manufacturing process via reactive distillation, including purification steps.

### BACKGROUND

Trifluoroacetyl iodide (CF₃COI) is a compound that can be converted to trifluoroiodomethane (CF₃I). Trifluoroiodomethane (CF₃I), also known as perfluoromethyliodide, trifluoromethyl iodide, or iodotrifluoromethane, is a useful compound in commercial applications as a refrigerant or a fire suppression agent, for example. Trifluoroiodomethane is a low global warming potential molecule with negligible ozone depletion potential. Trifluoroiodomethane can replace more environmentally damaging materials.

Methods of preparing trifluoroacetyl iodide are known. For example, the article, “The Reactions of Metallic Salts of Acids with Halogens. Part I. The Reaction of Metal Trifluoroacetates with Iodine, Bromine, and Chlorine,” R. N. Haszeldine, Journal of the Chemical Society, pp. 584-587 (1951), describes a batch reaction of trifluoroacetyl chloride and anhydrous hydrogen iodide without a catalyst for 8 hours at 120° C. to produce trifluoroacetyl iodide at a yield of about 62%. The poor yield and lengthy reaction times make it quite inefficient.

U.S. Pat. No. 7,196,236 (Mukhopadhyay et al.) discloses a catalytic process for producing trifluoroiodomethane using reactants comprising a source of iodine, at least a stoichiometric amount of oxygen, and a reactant CF₃R, where R is selected from the group consisting of —COOH, —COX, —CHO, —COOR₂, AND —SO₂X, where R₂ is alkyl group and X is a chlorine, bromine, or iodine. Hydrogen iodide, which may be produced by the reaction, can be oxidized by at least a stoichiometric amount of oxygen, producing water and iodine for economic recycling.

U.S. Pat. No. 7,132,578 (Mukhopadhyay et al.) also discloses a catalytic, one-step process for producing trifluoroiodomethane from trifluoroacetyl chloride. However, the source of iodine is iodine fluoride (IF). In contrast to hydrogen iodide, iodine fluoride is relatively unstable, decomposing above 0° C. to I₂ and IF₅. Iodine fluoride may also not be available in commercially useful quantities.

Some known methods of preparing trifluoroacetyl iodide include liquid-phase processes. Liquid-phase processes can require solvents that must be separated out and disposed of. The extra steps required for separation and disposal make the processes less efficient.

Thus, there is a need to develop a more efficient process that may be scaled to produce commercial quantities of trifluoroiodomethane from relatively inexpensive raw materials.

### SUMMARY

The present disclosure provides a process for making trifluoroacetyl iodide (TFAI) comprising: combining a stream comprising trifluoroacetyl chloride (TFAC) with a stream comprising hydrogen iodide (HI) to provide a combined reactant stream; passing the combined reactant stream to a reactive distillation column to provide a crude product stream; and purifying the crude product stream to provide a purified product stream comprising trifluoroacetyl iodide (TFAI). The reactive distillation may be performed in the presence of a catalyst. Alternatively, the reactive distillation may be performed in the absence of a catalyst. The process may be conducted in the presence of a solvent. Alternatively, the process may be conducted in the absence of an added solvent.

The above mentioned and other features of the disclosure, and the manner of attaining them, will become more apparent and will be better understood by reference to the following description of embodiments taken in conjunction with the accompanying drawings.

### BRIEF DESCRIPTION OF THE DRAWINGS

FIG. 1 shows a process flow diagram for an embodiment without a catalyst.

FIG. 2 shows a process flow diagram for an embodiment with a catalyst.

FIG. 3 shows a process flow diagram for an alternative embodiment without a catalyst.

FIG. 4 shows a process flow diagram for an alternative embodiment with a catalyst.

### DETAILED DESCRIPTION

The present disclosure provides continuous processes for producing trifluoroacetyl iodide (TFAI) from trifluoroacetyl chloride (TFAC) and hydrogen iodide. Specifically, the present disclosure provides a process for producing TFAI via a continuous reactive distillation process. The present disclosure further provides an integrated manufacturing process for the production of TFAI via reactive distillation, including purification steps.

As disclosed herein, TFAI may be produced according to the reaction shown below in Equation 1.

TFAC+HI→TFAI+HCl Eq. 1:

The reaction may be performed in the presence or absence of a catalyst. Briefly, TFAC and HI may be combined and vaporized before undergoing reactive distillation. During the reactive distillation process, both vapor phase and liquid phase reactions may occur. The vapor phase reaction may occur as the reactants boil, while the liquid phase reaction may occur as the reactants condense on the column packing. Continuous removal of HCl from the reaction may push the reaction to the right under thermodynamic equilibrium conditions, thereby limiting the reverse reaction.

Likewise, the reaction may be performed in the presence or absence of a solvent. When a solvent is present, it may be used to dissolve possible byproducts of the reaction, such as iodine for example. Methods both with and without a catalyst, as well as methods both with and without additional solvent, are described in greater detail below.

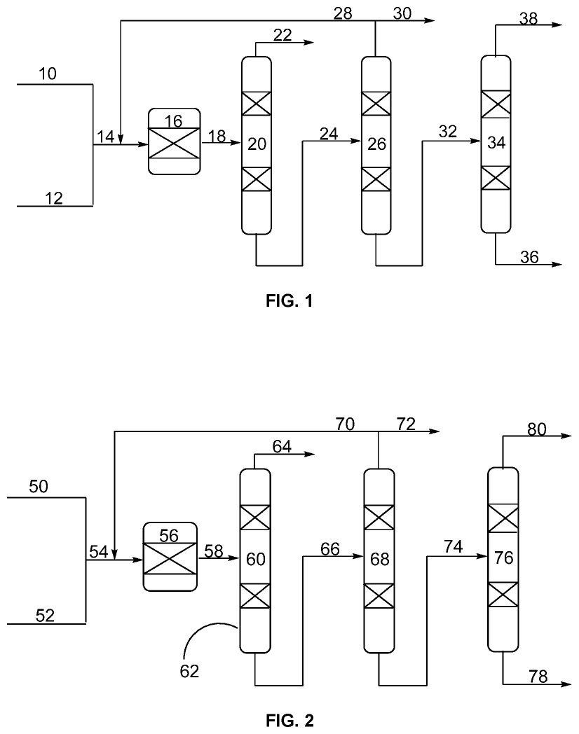

In one method, the reaction is performed without a catalyst as shown in FIG. 1. Liquid TFAC may be continuously added to a TFAC preheater (not shown) from a TFAC feed tank (not shown) to provide a reactant stream 10 comprising TFAC. Liquid HI may be continuously added to an HI preheater (not shown) from an HI feed tank (not shown) to provide a reactant stream 12 comprising HI. A feed stream 14 comprising a mixture of TFAC and HI may then be passed to a feed vaporizer 16 before being passed as vapor 18 to a packed reactive distillation column 20. Within the packed reactive distillation column 20, a vapor phase reaction may occur between TFAC and HI to form TFAI and HCl. As the TFAC and HI contact the packing in reactive distillation column 20 and condense, a liquid phase reaction may also occur between TFAC and HI to form TFAI and HCl. A liquid phase reaction between TFAC and HI may also occur in the column reboiler to form TFAI and HCl. From reactive distillation column 20, a first overhead stream 22 comprising HCl, hydrogen (H₂) and trifluoroacetyl fluoride (TFAF) may be removed for recovery. A first bottom stream 24 comprising unreacted TFAC, unreacted HI, R133 isomers (C₂H₂F₃Cl), trifluoroacetic acid (TFA), iodine, and other impurities, in addition to the desired TFAI product, may be fed to a second distillation column 26, also referred to as a TFAC/HI recycle column. From distillation column 26, a second overhead stream 28 comprising TFAC and HI with trace amount of HCl and TFAI may be recycled back to feed stream 14. Low-boiling impurities, such as R133 isomers, may be purged periodically as stream 30 from overhead stream 28. A second bottoms stream 32 comprising TFAI and high-boiling impurities such as TFA is continuously removed from distillation column 26 and passed to a third distillation column 34. A third bottoms stream 36 comprising high-boiling impurities such as TFA may be purged periodically. A third overhead stream 38 comprising purified TFAI may be collected.

Alternatively, the process may be conducted in the presence of a catalyst, as shown in FIG. 2. Liquid TFAC may be continuously added to a TFAC preheater (not shown) from a TFAC feed tank (not shown) to provide a reactant stream 50 comprising TFAC. Liquid HI may be continuously added to an HI preheater (not shown) from an HI feed tank (not shown) to provide a reactant stream 52 comprising HI. A feed stream 54 comprising a mixture of TFAC and HI may then be passed to a feed vaporizer 56 before being passed as vapor 58 to a packed reactive distillation column 60. The packed reactive distillation column may include a catalyst 62 at the bottom of the column packing. Within the packed reactive distillation column 60, a vapor phase reaction may occur between TFAC and HI as the reactants contact the catalyst 62, forming TFAI and HCl. As the TFAC and HI contact the packing in reactive distillation column 60, condense, and contact the catalyst 62, a liquid phase reaction may also occur between TFAC and HI to form TFAI and HCl. A liquid phase reaction between TFAC and HI may also occur in the column reboiler to form TFAI and HCl. From reactive distillation column 60, a first overhead stream 64 comprising HCl, hydrogen (H₂) and trifluoroacetyl fluoride (TFAF) may be removed for recovery. A first bottom stream 66 comprising unreacted TFAC, unreacted HI, R133 isomers (C₂H₂F₃Cl), trifluoroacetic acid (TFA), iodine, and other impurities, in addition to the desired TFAI product, may be fed to a second distillation column 68 also referred to as a TFAC/HI recycle column. From distillation column 68, a second overhead stream 70 comprising TFAC and HI with trace amount of HCl and TFAI may be recycled back to feed stream 54. Low-boiling impurities, such as R133 isomers, may be purged periodically as stream 72 from overhead stream 70. A second bottoms stream 74 comprising TFAI and high-boiling impurities such as TFA is continuously removed from distillation column 68 and passed to a third distillation column 76. A third bottoms stream 78 comprising high-boiling impurities such as TFA may be purged periodically. A third overhead stream 80 comprising purified TFAI may be collected.

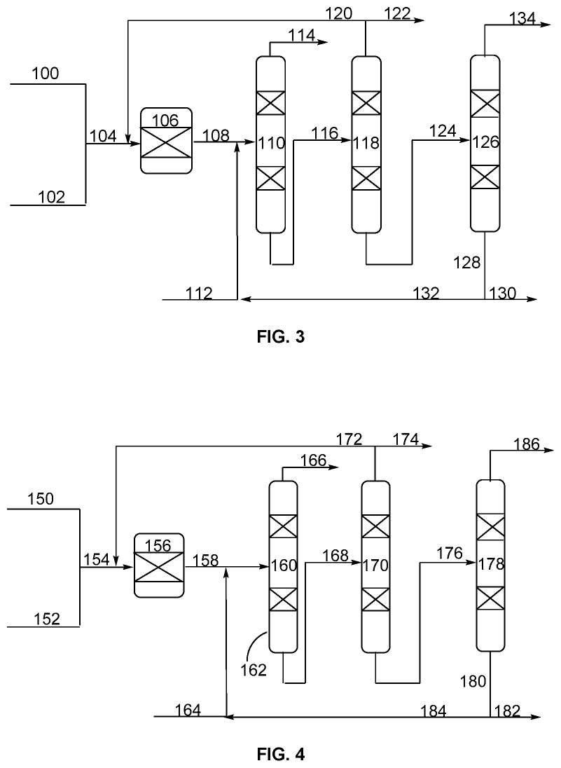

As yet another alternative, a high-boiling solvent may be introduced into the reactive distillation column. This may be desirable in some instances to dissolve solid iodine (I₂) which may form when TFAI is subjected to high temperatures and/or be carried over from HI feed. The presence of solid iodine may lead to lowered yields and difficulties with equipment plugging. The addition of a high-boiling solvent to dissolve solid iodine may eliminate plugging of equipment and permit for the recovery of iodine. As shown in FIG. 3, the reaction is conducted in the absence of a catalyst. Liquid TFAC may be continuously added to a TFAC preheater (not shown) from a TFAC feed tank (not shown) to provide a reactant stream 100 comprising TFAC. Liquid HI may be continuously added to an HI preheater (not shown) from an HI feed tank (not shown) to provide a reactant stream 102 comprising HI. A feed stream 104 comprising a mixture of TFAC and HI may then be passed to a feed vaporizer 106 before being passed as vapor 108 to a packed reactive distillation column 110. A high-boiling solvent 112 may be added to the packed reactive distillation column 110. Within the packed reactive distillation column 110, a vapor phase reaction may occur between TFAC and HI to form TFAI and HCl. As the TFAC and HI contact the packing in reactive distillation column 110 and condense, a liquid phase reaction may also occur between TFAC and HI to form TFAI and HCl. A liquid phase reaction between TFAC and HI may also occur in the column reboiler to form TFAI and HCl. From reactive distillation column 110, a first overhead stream 114 comprising HCl, hydrogen (H₂) and trifluoroacetyl fluoride (TFAF) may be removed for recovery. A first bottom stream 116 comprising the solvent, unreacted TFAC, unreacted HI, R133 isomers (C₂H₂F₃Cl), trifluoroacetic acid (TFA), iodine, and other impurities, in addition to the desired TFAI product, may be fed to a second distillation column 118 also referred to as a TFAC/HI recycle column. From distillation column 118, a second overhead stream 120 comprising TFAC and HI with trace amount of HCl and TFAI may be recycled back to feed stream 104. Low-boiling impurities, such as R133 isomers, may be purged periodically as stream 122 from overhead stream 120. A second bottoms stream 124 comprising TFAI, the solvent, iodine, and high-boiling impurities such as TFA is continuously removed from distillation column 118 and passed to a third distillation column 126. A third bottom stream 128 comprising the solvent, iodine, and high-boiling impurities such as TFA may be purged periodically as stream 130 to remove impurities and recover iodine. A bottom stream 132 comprising the solvent, TFAI, iodine, and high-boiling impurities such as TFA may be recycled back to the reactive distillation column feed stream 108. A third overhead stream 134 comprising purified TFAI may be collected.

As still another alternative, the method as shown in FIG. 3 may be modified to include a catalyst. As shown in FIG. 4, liquid TFAC may be continuously added to a TFAC preheater (not shown) from a TFAC feed tank (not shown) to provide a reactant stream 150 comprising TFAC. Liquid HI may be continuously added to an HI preheater (not shown) from an HI feed tank (not shown) to provide a reactant stream 152 comprising HI. A feed stream 154 comprising a mixture of TFAC and HI may then be passed to a feed vaporizer 156 before being passed as vapor 158 to a packed reactive distillation column 160. The packed reactive distillation column may include a catalyst 162 at the bottom of the column packing. A high-boiling solvent 164 may be added to the packed reactive distillation column 160. Within the packed reactive distillation column 160, a vapor phase reaction may occur between TFAC and HI as the reactants contact the catalyst 162, forming TFAI and HCl. As the TFAC and HI contact the packing in reactive distillation column 160, condense, and contact the catalyst 162, a liquid phase reaction may also occur between TFAC and HI to form TFAI and HCl. A liquid phase reaction between TFAC and HI may also occur in the column reboiler to form TFAI and HCl. From reactive distillation column 160, a first overhead stream 166 comprising HCl, hydrogen (H₂) and trifluoroacetyl fluoride (TFAF) may be removed for recovery. A first bottom stream 168 comprising the solvent, unreacted TFAC, unreacted HI, R133 isomers (C₂H₂F₃Cl), trifluoroacetic acid (TFA), iodine, and other impurities, in addition to the desired TFAI product, may be fed to a second distillation column 170 also referred to as a TFAC/HI recycle column. From distillation column 170, a second overhead stream 172 comprising TFAC and HI with trace amount of HCl and TFAI may be recycled back to feed stream 154. Low-boiling impurities, such as R133 isomers, may be purged periodically as stream 174 from overhead stream 172. A second bottoms stream 176 comprising TFAI, the solvent, iodine, and high-boiling impurities such as TFA is continuously removed from distillation column 170 and passed to a third distillation column 178. A third bottom stream 180 comprising the solvent, iodine, and high-boiling impurities such as TFA may be purged periodically as stream 182 to remove impurities and recover iodine. A bottom stream 184 comprising the solvent, TFAI, iodine, and high-boiling impurities such as TFA may be recycled back to the reactive distillation column feed stream 158. A third overhead stream 186 comprising purified TFAI may be collected.

Fresh HI and TFAC may be combined with a recycle mixture comprising HI and TFAC recovered from processing downstream, for example from a distillation train. The combined TFAC:HI molar ratio is provided with an excess of TFAC to give high conversion of the more expensive HI, although equimolar amounts or an excess of HI may also be used.

The molar ratio of TFAC:HI is about 1:10 or greater, about 1:5 or greater, about 1:2 or greater, about 1:1.9 or greater, about 1:1.8 or greater, about 1:1.7 or greater, about 1:1.6 or greater, about 1:1.5 or greater, about 1:1.4 or greater, about 1:1.3 or greater, about 1:1.2 or greater, about 1:1.1 or greater, about 1:1 or less, about 1.1:1 or less, about 1.2:1 or less, about 1.3:1 or less, about 1.4:1 or less, about 1.5:1 or less, about 1.6:1 or less, about 1.7:1 or less, about 1.8:1 or less, about 1.9:1 or less, about 2:1 or less, about 5:1 or less, about 10:1 or less, or any value or range encompassed by these endpoints. Preferably, the molar ratio of TFAC:HI is from about 1:1 to about 2:1.

The reactive distillation column reboiler temperature may be about 20° C. or higher, about 30° C. or higher, about 40° C. or higher, about 50° C. or higher, about 60° C. or higher, about 70° C. or lower, about 80° C. or lower, about 90° C. or lower, about 100° C. or lower, about 110° C. or lower, about 120° C. or lower, or any value or range encompassed by these endpoints. Preferably, the temperature is from about 50° C. to about 110° C.

The temperature of the overhead stream from the reactive distillation column may be about −60° C. or higher, about −50° C. or higher, about −40° C. or higher, about −30° C. or higher, about −20° C. or lower, about −10° C. or lower, about 0° C. or lower, about 10° C. or lower, or any value or range encompassed by these endpoints.

The reactive distillation column may be operated at a pressure of about 10 psig or higher, about 20 psig or higher, about 30 psig or higher, about 40 psig or higher, about 50 psig or higher, about 60 psig or higher, about 70 psig or higher, about 100 psig or higher, about 200 psig or lower, about 300 psig or lower, about 400 psig or lower, about 500 psig or lower, or any value or range encompassed by these endpoints. Preferably, the pressure is from about 50 to 100 psig.

The temperature of the TFAC/HI recycle column overhead stream may be about −50° C. or higher, about −40° C. or higher, about −30° C. or higher, about −20° C. or higher, about −10° C. or higher, about 0° C. or higher, about 10° C. or lower, about 20° C. or lower, about 30° C. or lower, about 40° C. or lower, about 50° C. or lower, about 60° C. or lower, about 70° C. or lower, about 80° C. or lower, or any value or range encompassed by these endpoints.

The temperature of the TFAC/HI recycle column bottom stream may be about 50° C. or higher, about 60° C. or higher, about 70° C. or higher, about 80° C. or higher, about 90° C. or higher, about 100° C. or lower, about 110° C. or lower, about 120° C. or lower, about 130° C. or lower, about 140° C. or lower, about 150° C. or lower, or any value or range encompassed by these endpoints.

The TFAC/HI recycle column may be operated at a pressure of about 10 psig or higher, 14 psig or higher, about 20 psig or higher, about 30 psig or higher, about 40 psig or higher, about 50 psig or higher, about 60 psig or lower, about 70 psig or lower, about 80 psig or lower, about 90 psig or lower, about 100 psig or lower, or any value or range encompassed by these endpoints. Preferably, the pressure is about 10 psig to about 30 psig.

When a catalyst is used, it may be selected from the group comprising activated carbon, meso carbon, stainless steel, nickel, nickel-chromium alloy, nickel-chromium-molybdenum alloy, nickel-copper alloy, copper, alumina, platinum, palladium, or carbides, such as metal carbides, such as iron carbide, molybdenum carbide and nickel carbide, and non-metal carbides, such as silicon carbide, or combinations thereof.

The catalyst may be in the form of a mesh, pellet, or sphere.

When a solvent is used, it is desirable that the solvent displays high iodine solubility. Suitable solvents may include benzene and substituted aromatics such as toluene, xylenes, mesitylene (1,3,5-trimethylbenzene), ethyl benzene and the like; polar aprotic solvents such as dimethylformamide (DMF) and dimethyl sulfoxide (DMSO); and ionic liquids such as imidazolium salts and caprolactamium hydrogen sulfate, for example; and combinations thereof.

The process may produce trifluoroacetyl iodide (TFAI) in a yield of about 85% or greater, about 90% or greater, about 95% or greater, about 96% or greater, about 97% or greater, about 98% or greater, or about 99% or greater.

The trifluoroacetyl iodide (TFAI) may be produced in a purity of about 90% or greater, about 91% or greater, about 92% or greater, about 93% or greater, about 94% or greater, about 95% or greater, about 96% or greater, about 97% or greater, about 98% or greater, or about 99% or greater.

While this invention has been described as relative to exemplary designs, the present invention may be further modified within the spirit and scope of this disclosure. Further, this application is intended to cover such departures from the present disclosure as come within known or customary practice in the art to which this invention pertains.

As used herein, the phrase “within any range defined between any two of the foregoing values” literally means that any range may be selected from any two of the values listed prior to such phrase regardless of whether the values are in the lower part of the listing or in the higher part of the listing. For example, a pair of values may be selected from two lower values, two higher values, or a lower value and a higher value.

### EXAMPLES

### Example 1: Formation of TFAI in the Absence of a Catalyst

This example illustrates a continuous process to produce TFAI from TFAC and HI via reactive distillation in the absence of a catalyst, as well as the purification of TFAI from reaction products. The unit consists of a TFAC and HI feed system, a TFAC/HI feed mixer/vaporizer, a reactive distillation column, a second distillation column which acts as a TFAC/HI recycle column and a third distillation column which acts as a TFAI purification column. All three columns have the same setup, consisting of a 10 gallon reboiler, a 2″ ID×120″ long column with Goodloe 2″ diameter×6″ thick structured metal packing and a tube in shell condenser.

392 g/h of liquid TFAC and 379 g/h of liquid HI are fed into the reactive distillation column with the column reboiler temperature controlled at about 56° C. by saturated steam at 30 psig, and the column overhead temperature controlled at −47° C. with the condenser cooled by liquid nitrogen. About 107 g/h of HCl is removed from the column overhead to the KOH scrubber system with the column overhead pressure controlled at 70 psig. By IC analysis, the HCl stream contains mainly HCl with trace amount of TFAF. The reactive distillation column bottoms stream is fed into the second column with the column reboiler temperature controlled at about 65° C. by saturated steam at 30 psig, and the column overhead temperature controlled at about 8° C. with the condenser cooled by chilled ethanol. With the overhead pressure controlled at 28 psig, 210 g/h of the second column overhead stream containing unreacted TFAC (88.1 wt. %) and HI (9.5 wt. %), HCl (<0.5 wt. %) and TFAI (<2%), balanced with others, are recycled back to the feed vaporizer. Periodically, the second column overhead stream can be purged if low-boiling impurities, such as R133 isomers, are accumulated. The second column bottoms stream comprising mainly TFAI with <1 wt. % TFA and other heavies is fed into the third distillation column, wherein the column reboiler temperature is controlled at about 74° C. by saturated steam at 30 psig, and the column overhead temperature controlled at 36° C. with the condenser cooled by city water. With the second column overhead pressure at ambient, about 650 g/h of pure TFAI (>99 wt. %) is collected from the column overhead stream, representing a TFAI yield of >97%. Periodically, the third column bottoms stream can be purged if high-boiling impurities, such as iodine and TFA are accumulated.

### Example 2: Formation of TFAI in the Presence of a Catalyst

This example illustrates a continuous process to produce TFAI from TFAC and HI via reactive distillation in the presence of a catalyst, as well as the purification of TFAI from reaction products. The unit consists of a TFAC and HI feed system, a TFAC/HI feed mixer/vaporizer, a reactive distillation column, a second distillation column which acts as a TFAC/HI recycle column and a third distillation column which acts as a TFAI purification column. The reactive distillation column consists of a 10 gallon reboiler, a 2″ ID×120″ long column and a tube in shell condenser. The column is packed with granular activated carbon (6″ thick, 309 ml by volume) at the bottom which is served as the catalyst, with the rest of the column packed with Goodloe 2″ diameter×6″ thick structured metal packing. The other two columns have the same setup, consisting of a 10 gallon reboiler, a 2″ ID×120″ long column with Goodloe 2″ dia×6″ thick structured metal packing and a tube in shell condenser.

359 g/h of liquid TFAC and 347 g/h of liquid HI are fed into the reactive distillation column wherein the column reboiler temperature controlled at about 59° C. by saturated steam at 30 psig, and the column overhead temperature controlled at −47° C. with the condenser cooled by liquid nitrogen. About 99 g/h of HCl is removed from the column overhead to the KOH scrubber system with the column overhead pressure controlled at 70 psig. By IC analysis, the HCl stream contains mainly HCl with trace amount of TFAF. The reactive distillation column bottoms stream is fed into the second column with the column reboiler temperature controlled at about 65° C. by saturated steam at 30 psig, and the column overhead temperature controlled at about 8° C. with the condenser cooled by chilled ethanol. With the overhead pressure controlled at 28 psig, 164 g/h of the second column overhead stream comprising unreacted TFAC (94.1 wt. %) and HI (4.3 wt. %), HCl (<0.3 wt. %) and TFAI (<1.5%), balanced with others, are recycled back to the feed vaporizer. Periodically, the second column overhead stream can be purged if low-boiling impurities, such as R133 isomers, are accumulated. The second column bottoms stream comprising mainly TFAI with <1 wt. % TFA and other heavies is fed into the third distillation column with the column reboiler temperature controlled at about 74° C. by saturated steam at 30 psig, and the column overhead temperature controlled at 36° C. with the condenser cooled by city water. With the second column overhead pressure at ambient, about 598 g/h of pure TFAI (>99 wt. %) is collected from the column overhead stream, representing a TFAI yield of >97%. Periodically, the third column bottoms stream can be purged if high-boiling impurities, such as iodine, TFA are accumulated.

### Example 3: Formation of TFAI with Addition of a Solvent

With the same setup and operation as described in Example 1, two kg of toluene is added into the reactive distillation reboiler at the start-up of the operation. The addition of toluene to the system serves as a solvent to dissolve solid iodine (I₂) which may form when TFAI is subjected to high temperatures and/or be carried over from HI feed. The toluene is passed through the system from the reactive distillation column bottoms to the second distillation column, then from the second distillation column bottoms to the third distillation column, and recycled back to the reactive distillation column reboiler from the third distillation column bottoms. When iodine concentration reaches about 20 wt. %, or TFA concentration reaches about 30 wt. %, a certain amount of reboiler material is purged out from the third distillation column bottoms stream, and an approximately equivalent amount of fresh toluene is added to the reactive distillation reboiler to make up the toluene being purged. The purged toluene stream can be treated to recover toluene and iodine for recycle or disposed of as waste.

### Example 4: Formation of TFAI in the Presence of a Catalyst with Addition of a Solvent

With the same setup and operation as described in Example 2, two kg of toluene is added into the reactive distillation reboiler at the start-up of the operation. The addition of toluene to the system serves as a solvent to dissolve solid iodine (I₂) which may form when TFAI is subjected to high temperatures and/or be carried over from HI feed. The toluene is passed through the system from the reactive distillation column bottoms to the second distillation column, then from the second distillation column bottoms to the third distillation column, and recycled back to the reactive distillation column reboiler from the third distillation column bottoms. When iodine concentration reaches about 20 wt. %, or TFA concentration reaches about 30 wt. %, a certain amount of reboiler material is purged out from the third distillation column bottoms stream, and about equivalent amount of fresh toluene is added to the reactive distillation reboiler to make up the toluene being purged. The purged toluene stream can be treated to recover toluene and iodine for recycle or disposed of as waste.

### Aspects

Aspect 1 is a process for making trifluoroacetyl iodide (TFAI), the process comprising: combining a stream comprising trifluoroacetyl chloride (TFAC) with a stream comprising hydrogen iodide (HI) to provide a combined reactant stream; passing the combined reactant stream to a reactive distillation column to provide a crude product stream; and purifying the crude product stream to provide a purified product stream comprising trifluoroacetyl iodide (TFAI).

Aspect 2 is the process of Aspect 1, wherein the molar ratio of TFAC:HI is about 1:1 to about 2:1.

Aspect 3 is the process of either Aspect 1 or Aspect 2, wherein the reactive distillation column is a packed column further comprising a reboiler.

Aspect 4 is the process of any one of Aspects 1 to 3, wherein the reactive distillation column reboiler temperature is about 50° C. to 110° C.

Aspect 5 is the process of any one of Aspects 1 to 4, wherein the reactive distillation column is operated at a pressure of about 50 to 100 psig.

Aspect 6 is the process of any one of Aspects 1 to 5, wherein purifying the crude product stream comprises passing the crude product stream to one or more distillation columns.

Aspect 7 is the process of any one of Aspects 1 to 6, wherein the reactive distillation column further comprises a catalyst.

Aspect 8 is the process of Aspect 7, wherein the catalyst is selected from the group consisting of activated carbon, meso carbon, stainless steel, nickel, nickel-chromium alloy, nickel-chromium-molybdenum alloy, nickel-copper alloy, copper, alumina, platinum, palladium, iron carbide, molybdenum carbide, nickel carbide, silicon carbide, and combinations thereof.

Aspect 9 is the process of any one of Aspects 1 to 8, wherein the combined reactant stream further comprises a solvent.

Aspect 10 is the process of Aspect 9, wherein the solvent is selected from the group consisting of benzene, toluene, xylenes, mesitylene (1,3,5-trimethylbenzene), ethyl benzene, dimethylformamide (DMF), dimethyl sulfoxide (DMSO), ionic liquids, and combinations thereof.

Aspect 11 is the process of either Aspect 9 or Aspect 10, further comprising recovering iodine (I₂) from the purified product stream.

What is claimed is:

1. A process for making trifluoroacetyl iodide (TFAI), the process comprising:
combining a stream comprising trifluoroacetyl chloride (TFAC) with a stream comprising hydrogen iodide (HI) to provide a combined reactant stream;
passing the combined reactant stream to a reactive distillation column to provide a crude product stream; and
purifying the crude product stream to provide a purified product stream comprising trifluoroacetyl iodide (TFAI).

2. The process of claim 1, wherein the molar ratio of TFAC:HI is about 1:1 to about 2:1.

3. The process of claim 1, wherein the reactive distillation column is a packed column further comprising a reboiler.

4. The process of claim 1, wherein the reactive distillation column reboiler temperature is about 50° C. to 110° C.

5. The process of claim 1, wherein the reactive distillation column is operated at a pressure of about 50 to 100 psig.

6. The process of claim 1, wherein purifying the crude product stream comprises passing the crude product stream to one or more distillation columns.

7. The process of claim 1, wherein the reactive distillation column further comprises a catalyst.

8. The process of claim 7, wherein the catalyst is selected from the group consisting of activated carbon, meso carbon, stainless steel, nickel, nickel-chromium alloy, nickel-chromium-molybdenum alloy, nickel-copper alloy, copper, alumina, platinum, palladium, iron carbide, molybdenum carbide, nickel carbide, silicon carbide, and combinations thereof.

9. The process of claim 1, wherein the combined reactant stream further comprises a solvent.

10. The process of claim 9, wherein the solvent is selected from the group consisting of benzene, toluene, xylenes, mesitylene (1,3,5-trimethylbenzene), ethyl benzene, dimethylformamide (DMF), dimethyl sulfoxide (DMSO), ionic liquids, and combinations thereof.

11. The process of claim 9, further comprising recovering iodine (I₂) from the purified product stream.
